(19) United States
(12) Patent Application Publication
(10) Pub. No.: US 2007/0293754 A1
(43) Pub. Date: Dec. 20, 2007

(54) Probe With Asymmetric Balloon

(76) Inventors: Nachum Schneid, Lod (IL); Gadi Lewkonya, Neve Mivtach (IL); Yoav Venkert, Lod (IL); Avner Bar Lev, Lod (IL)

Correspondence Address: Martin D Moynihan;Prtsi, P.O Box16446, Arlington, VA, 22215, US

(21) Appl. No.: 11/665,699

(22) PCT Filed: Oct. 17, 2005
(86) PCT No.: PCT/IL05/01098

§ 371 Date: Apr. 18, 2007

Related U.S. Application Data
(63) Continuation-in-part of application No. 10/968,853, filed on Oct. 18, 2004.
(63) Continuation-in-part of application No. PCT/IL2005/000074, filed on Jan. 20, 2005.

(51) Int. Cl. A61B 5/055

(52) U.S. Cl. 600/423

(57) ABSTRACT

A device adapted to be inserted into a lumen, the device having a longitudinal axis and comprising: a) a support element extending along the longitudinal axis; b) a tool mechanically mounted on the support element and being adapted to be used near a wall of the lumen on at least a first side of the longitudinal axis; and c) element, the balloon having at least one portion that is less radially expandable than at least one other portion of the balloon, at a same axial position along said support element.

### RELATED APPLICATIONS

The present application is a continuation-in-part of U.S. Ser. No. 10/968,853, entitled “Magnet and Coil Configurations for MRI Probes”, filed on Oct. 18, 2004, and is a continuation in part of PCT/IL2005/000074, entitled “MRI Probe for Prostate Imaging”, filed on Jan. 20, 2005, the disclosures of which are incorporated herein by reference.

### FIELD OF THE INVENTION

The field of the invention is diagnostic and therapeutic probes, including medical probes.

### BACKGROUND OF THE INVENTION

A variety of medical diagnostic and therapeutic probes, used in blood vessels and other lumens inside the body, include expandable balloons, either to temporarily hold the probe in place, or to enlarge the lumen. For example, balloons are commonly used to expand stents used for angioplasty in arteries. Balloons used in angioplasty are typically made of a thick, rather inelastic material, and are inserted into the artery in a folded state. They unfold as they are expanded, typically by injecting saline solution into them under pressure. Once the pressure is released and they collapse, they do not refold themselves, so they cannot easily be moved to a different location and re-expanded. Since these balloons are relatively inelastic, a given balloon is only designed for use in a narrow range of lumen diameters, for example, within 10% of the nominal expansion diameter. However, there are some medical applications where elastic balloons are used, which can be expanded to a relatively large range of diameters by using different pressures.

Most balloons used in medical probes are axisymmetric and have uniform elasticity. For example, Golan in U.S. Pat. No. 6,600,319, and Blank et al in U.S. Pat. No. 6,704,594, the disclosures of which are incorporated herein by reference, each describe a self-contained intravascular MRI probe which is held in place in the center of a blood vessel by axisymmetric balloons, and is rotated to successively image different azimuthal sectors of the blood vessel wall, to detect plaque. Tu et al, in U.S. Pat. No. 6,036,689, the disclosure of which is incorporated herein by reference, describe an intravascular probe with RF electrodes, used for ablation of plaque, arranged in an expandable basket, with an axisymmetric balloon in the center. When the balloon expands, the basket expands, pushing the electrodes against the wall of the blood vessel, together with temperature sensors to provide feedback during the ablation process.

There are some medical devices which use balloons that are not uniformly elastic. Mikhail et al, in U.S. Pat. No. 5,707,357, the disclosure of which is incorporated herein by reference, describes a urinary catheter with an anchoring balloon that is axisymmetric, but has non-uniform thickness or non-uniform bonding patterns which alter the shape of the balloon when it is expanded. Richter, in PCT publication WO 01/95833 A3, the disclosure of which is incorporated herein by reference, describes two axisymmetric balloons, one inside the other, which are used to implant a stent. The inner balloon, which is shorter than the stent, is expanded first, expanding a center portion of the stent. The inner balloon then bursts, and the outer balloon, which is longer than the stent and has a much higher bursting pressure than the inner balloon, then expands, causing the rest of the stent to expand uniformly, and preventing an undesirable condition called “dogboning” in which the ends of a stent expand more than the center.

Brennan et al, in U.S. patent application publication U.S. 2003/0109810 A1, the disclosure of which is incorporated herein by reference, describes a guide catheter, the end of which is steered by inflating and deflating a balloon. The end of the catheter has a flexible shaft with a preformed bend, surrounded by an initially deflated balloon. When the balloon is expanded, it forces the preformed bend to straighten out partially, and when the balloon is deflated, the end of the catheter bends again.

Reilly et al, in U.S. Pat. No. 6,235,043, the disclosure of which is incorporated herein by reference, describes a non-axisymmetric balloon for insertion into a medullary cavity of a bone. The balloon is made of a non-elastic material, but is folded up, so that it can be inserted through a narrow tube.

Schnall et al, U.S. Pat. No. 5,476,095, the disclosure of which is incorporated herein by reference, describes an MRI receiver probe designed for prostate imaging in the rectum. The probe includes an asymmetric elastic balloon mounted on a probe shaft, and an MRI receiver antenna mounted on the inside of the balloon. When the balloon expands, the antenna moves away from the probe shaft to a position adjacent to the prostate.

Nohilly et al, U.S. published patent application 2005/0113857 A1, the disclosure of which is incorporated herein by reference, describes an elastic surgical balloon shaped to fill the inside of the uterus, and used for purposes such as thermal ablation of tissue in the wall of the uterus.

### SUMMARY OF THE INVENTION

An aspect of some embodiments of the invention concerns a probe, for example an intravascular probe, in which a non-axisymmetric elastically expandable balloon is mounted on a relatively rigid support element, which has a longitudinal axis aligned with a lumen such as a blood vessel. When the balloon expands, a more expandable part of the balloon, on a first side of the axis, expands further away from the support element, while a less expandable part of the balloon, on a second side of the axis, opposite to the first side, remains much closer to the support element. As a consequence, when the balloon is expanded sufficiently so that it reaches the wall of the blood vessel, it holds the support element in place closer to the wall on the second side than on the first side. A tool, optionally mechanically mounted on the support structure, is thus brought close to the wall of the blood vessel on the second side, where it optionally performs a diagnostic or therapeutic task, or both. As used herein, the term “diagnostic task” includes any kind of medical information gathering, not necessarily limited to final diagnosis of a medical condition. The tool is, for example, an MRI sensor which produces images of plaque in the wall.

As used herein, “mechanically mounted on the support structure” means that the support structure provides support for the tool. In some embodiments of the invention, the tool is mounted on the support structure on the second side of the axis. In some embodiments of the invention, the tool is rigidly mounted on the support structure, and substantially does not move relative to the support structure during the normal operation of the probe. Alternatively, the tool is flexibly mounted on the support structure, but is sufficiently well coupled to the support structure that it does not move freely with respect to the support structure. In some embodiments of the invention, one or more elements of the tool also function as the support structure, or as a part of the support structure. For example, magnets may function both as part of an MRI sensor tool, and as part of the support structure.

The asymmetric balloon brings the sensor closer to the wall than if an axisymmetric balloon were used which held the support element in the center of the blood vessel. Furthermore, a potential advantage of the balloon being elastic on one side is that the same probe can be used with a fairly wide range of blood vessel diameters, by applying different pressures to the fluid filling the balloon. In contrast, if a conventional inelastic angioplasty balloon were used, then the diameter of the fully inflated balloon would be nearly fixed, varying by perhaps 10% over the safe range of pressures, and the probe could only be used with a narrow range of blood vessel diameters. Another potential advantage of an elastic balloon is that the balloon may provide a greater safety margin against bursting, than an inelastic balloon.

Alternatively, such a balloon can be used for other types of sensors that are used close to a blood vessel wall, or for therapeutic devices which remove plaque, for example. A similar balloon may also be used in other lumens in the body, for example in the urethra, or in the digestive track, including, for example, in the rectum for prostate imaging, or any minimally invasive procedure. A similar balloon may also be used in more invasive procedures, for example in a breast biopsy, to hold a probe in place in a channel made as part of the procedure, as opposed to a pre-existing lumen.

In an exemplary embodiment of the invention, the balloon is stiffened by a stiffening material, for instance parylene, more in a first portion thereof than in other portions, such that in the stiffened portion the expandability of the balloon is smaller than in its other portions.

According to one embodiment of the invention, the asymmetrically expandable balloon is double walled, and the walls are attached to each other, for instance by heat fusing, in one portion. Thus, when the balloon is inflated, the portion with the walls that are attached to each other does not expand.

According to one embodiment of the invention, the balloon is wrapped with an outer layer held firmly to the support and having at least one opening. The outer layer inhibits the balloon's expansion, and thus expansion occurs mainly at the opening of the outer layer. The outer layer may be made of a shrinking material, for instance, heat shrinking, and be held firmly to the support by shrinking around it.

An aspect of the invention relates to manufacturing a balloon with non-axisymmetric expandability. According to one embodiment of the invention, a balloon of uniform elasticity, made for example from a tube of circular cross-section, is mounted on a support element. The balloon is masked asymmetrically, covering more of the area on one side of the axis of the support element than on the other side of the axis, or covering one side for more time than the other side. A stiffening material, for example parylene, is then deposited on the masked balloon, and the masking is removed. The balloon will be more elastic in the region which was masked, which will have less parylene, if any, while the unmasked region, covered with more parylene, will be less elastic.

According to some embodiments of the invention, the support is first wrapped with an inner layer. Then, the obtained wrapped support is further wrapped with an expandable layer, which is then made asymmetrically expandable, for example, by selective application of a stiffening material to one portion thereof, or by attaching one portion thereof to the inner layer, for instance by heat fusing, thereby inhibiting the expanding layer from expanding in that portion. In some embodiments of the invention, the expandable layer is wrapped with an outer layer with an opening in it, and the outer layer inhibits the expanding layer from expanding except through the opening.

According to one embodiment of the present invention, there is provided a device comprising an asymmetric balloon, MRI coil and magnet.

According to one embodiment of the present invention, there is provided a device adapted to be inserted into a lumen. The device has a longitudinal axis and comprises (i) a support element extending along the longitudinal axis; (ii) a tool being adapted to be used near a wall of the lumen on at least a first side of the longitudinal axis; and (iii) an elastically inflatable balloon mounted on the support element, optionally surrounding it and the tool. The balloon has at least one portion that is less radially expandable, at a given axial position along the support element, than at least one other portion of the balloon at the same axial position. In this embodiment, the tool is mechanically mounted on the support element. One way to mount the tool on the support element is to include a sealing element in the device which holds the balloon against the support element in a pressure-tight manner. It should be noted that the tool may be an integral part of the support, and in some embodiments, the tool may also function as a support element.

Optionally, the lumen is in the body. Non-limiting examples of such lumens are portions of the digestive track, the rectum, and blood vessels.

Optionally, the tool is configured to perform a diagnostic task, for example, a diagnostic task that includes imaging, information gathering by NMR, detecting plaque in a blood vessel, diagnosing one or more characteristics of plaque in a blood vessel, and/or others. Thus, non-limiting examples of tools are MRI coils and/or magnets.

Exemplary outer diameters of the device, when the balloon is not inflated, are diameters less than 1 mm, less than 2 mm, and between about 2 and about 4 mm.

Optionally, the balloon of the device safely inflates to a range of sizes varying by at least a factor of 2, and this range of sizes may contain 3 mm. The term safely inflates means that the danger of bursting during such inflation is within allowed limits according to medical standards acceptable for the specific use intended for the specific device.

The balloon may be made of elastic materials that comprise silicone, a blend of silicone and polyurethane, and others.

In an exemplary embodiment, the portion of the balloon that expands less is thicker than other portions.

In an exemplary embodiment, the balloon is coated with a stiffening material on at least the first side of the axis. Optionally, at least a portion of the balloon on the side of the axis opposite the first side is not coated with the stiffening material. A non-limiting example for a suitable stiffening material is parylene.

In an exemplary embodiment, a heat shrink material is applied to a balloon's portion that is less expandable than other portions of the balloon. Optionally, the heat shrink material comprises PET (polyethylene tetraphtalate).

In accordance with another embodiment of the present invention there is provided a method of manufacturing a device adapted to be inserted into a lumen. This method includes placing an elastically expandable balloon around a support element having an axis; masking the balloon asymmetrically, such that a first portion of the balloon is masked more than a second portion of the balloon, said first and second portions extending along the axis; applying a stiffening material, for example parylene, to the masked balloon; and unmasking the balloon. Optionally, the differences in amount of stiffening material applied to different portions of the balloon allow differences in expandability of such different portions, such that an inflated balloon has one portion that is expanded at least 35% more than another.

Optionally, applying a stiffening material in accordance with the invented method includes vapor depositing the stiffening material onto the masked balloon.

In accordance with an exemplary embodiment of the invention, there is provided a method of manufacturing a device adapted to be inserted into a lumen, the method comprising: placing an elastically expandable balloon around a rigid support element having an axis; applying a heat-shrink material to a first portion of the balloon; and heating the heat-shrink material as to let it shrink around the first portion of the balloon.

In accordance with an exemplary embodiment of the invention, there is provided a method of manufacturing a device adapted to be inserted into a lumen, the method comprising fixing a sheet of polymeric material around a support element having an axis; piercing this sheet as to allow an inflation tube extending along the support element to supply an inflating fluid through the pierce; placing an elastic balloon around the pierced sheet; and heat-fusing a first portion of the elastic balloon to an adjacent portion of the pierced sheet, such that upon supplying inflating material through the inflation tube, the first portion of the balloon will not inflate. The first portion may, however, be distorted due to forces exerted on it by inflated portions adjacent to it. The sheet of polymeric material may include a blend of silicone and polyurethane, such as the blend commercially available under the name of Polyblend™. The elastic balloon may be made of the same material as the polymeric sheet.

The methods of the invention may also include sealing the balloon against the support element, for instance, by application of glue. Non-limiting examples of suitable kinds of glue are cyanoacrylates and UV-curable glue. In all methods of the invention, a tool is optionally mechanically mounted on the support element.

According to one embodiment of the present invention there is provided a method of performing a diagnostic task, such as diagnostic imaging, diagnostic NMR, plaque detection, and/or diagnosis of plaque characteristics. The task is performed on the wall of a lumen in the body, for instance, on the wall of a blood vessel. This method includes inserting a device according to the invention into the lumen; expanding the balloon of the device; and performing the diagnostic task on the wall of the lumen on the first side of the axis, using the device.

### BRIEF DESCRIPTION OF THE DRAWINGS

In order to better understand the invention and to see how it may be carried out in practice, exemplary embodiments of the invention are described in the following sections, by way of non-limiting examples only, with reference to the drawings, which are generally not to scale. Tools used for such purposes as punching, cutting, stretching, and injecting glue, shown in some of these drawings, are intended to be symbolic, and are not intended to illustrate realistic shapes and sizes for these tools. In some of these drawings, some elements are shown in a schematic perspective view, to show their azimuthal extent, rather than in a cross-sectional view.

FIG. 1A is a schematic cross-sectional view, parallel to the longitudinal axis, of an uninflated probe inside a blood vessel, according to an exemplary embodiment of the invention;

FIG. 1B is a view of the same probe when it is inflated inside the blood vessel;

FIGS. 2 through 20 are schematic cross-sectional views, parallel to the longitudinal axis, showing different stages in the assembly of a probe such as that shown in FIG. 1A and FIG. 1B, according to an exemplary embodiment of the invention;

FIGS. 21A and 21B are the upper and lower parts of a flow chart listing the steps illustrated in FIGS. 2 through 20.

FIGS. 22-24 schematically show, in a cross-sectional view, a preparation of a probe for assembly to a balloon, as carried out according to some exemplary embodiments of the invention;

FIGS. 25-34 schematically show, in a cross-sectional view, a preparation of a balloon and an assembly of the balloon to the probe shown in FIGS. 22-24, according to an exemplary embodiment of the invention;

FIGS. 35-37 schematically show a procedure for making a balloon, prepared and assembled to a probe as in the exemplary embodiments of FIGS. 22-24, asymmetrically expandable, according to an exemplary embodiment of the invention;

FIGS. 38A-38C schematically show procedures used for making a balloon, prepared and assembled to a probe, asymmetrically expandable, according to another exemplary embodiment of the invention; and

FIGS. 39A and 39B schematically show side cross-sectional and axial cross-sectional views, respectively, of an axisymmetric balloon assembled to a probe, made according to one of the procedures shown in FIGS. 38A-38C.

### DETAILED DESCRIPTION OF EXEMPLARY EMBODIMENTS

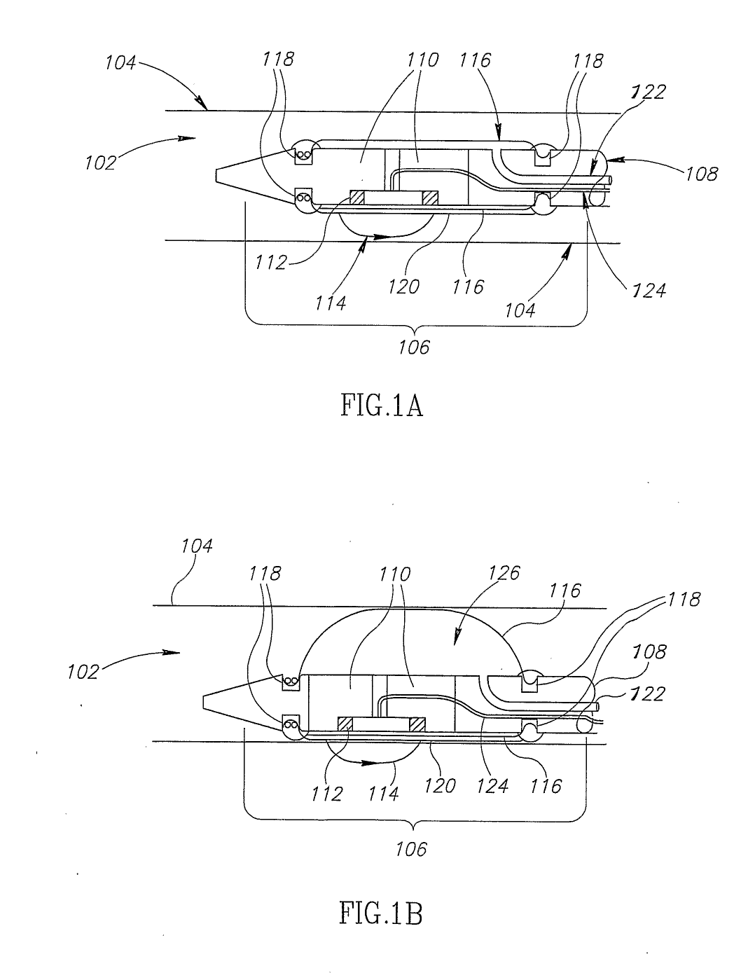

FIG. 1A shows a longitudinal cross-sectional view of a blood vessel 102 surrounded by a wall 104, with a probe 106 inside it, in accordance with one embodiment of the invention. Probe 106 is located at the end of a catheter 108 inside the blood vessel. Optionally, much of the volume of the probe is taken up by permanent magnets 110 and an RF coil 112 used to produce MRI images of the blood vessel wall, particularly to locate plaque. Magnets 110 produce magnet field lines, typified by field line 114, in the region to be imaged, while RF coil 112 produces a sequence of RF pulses in the imaging region, and receives NMR signals emitted by excited nuclei in the imaging region, which are used to reconstruct MRI images. Optionally, probe 106 also contains electronic components, for example a tuning capacitor, which may work better if they are located close to the probe.

In order to hold probe 106 against the blood vessel wall, so that the imaging region of the probe extends into the wall, and so that the probe does not move relative to the wall while an image is acquired, there is an elastic balloon 116 which surrounds a central portion of probe 106 (i.e. the support element referred to in the Summary) and whose ends are sealed against the probe. The bottom side of balloon 116, as shown in FIG. 1A, is coated with a layer 120 of parylene, which makes the bottom side of the balloon inelastic. The orientation shown in the figure was selected for convenience only, and other orientations are also in accordance with the invention. The advantages of the invention are, however, most pronounced when the MRI probe is at the less expandable side of the probe.

In an exemplary embodiment of the invention, inflation is by injecting a biologically safe fluid such as saline solution under pressure through an inflation tube 122 into balloon 116. The top part of balloon 116, which is optionally not coated with parylene, is elastic and expands, as shown in FIG. 1B, where interior region 126 of the balloon is filled with pressurized fluid. The bottom part of balloon 116, coated with a stiffening material such as parylene, is much less elastic and hardly changes shape when the pressurized fluid is injected. Fluid is injected with sufficient pressure to expand the balloon until the probe reaches opposite walls of the blood vessel. The bottom part of the probe is then held firmly in place against the bottom wall 104 of the blood vessel. Optionally, the resulting difficulty in moving the probe is used to verify that the balloon is fully expanded, and there is no need to raise the pressure of the expansion fluid. Additionally or alternatively, a fluoroscope or other imaging means is used to verify that the balloon is sufficiently expanded to hold the probe firmly in place, or the MRI data itself is used to verify this, for example by seeing that the MRI signal from the blood vessel wall indicates that the blood vessel wall is in contact with the probe. Magnets 110 are then close enough to the blood vessel wall, such that the blood vessel wall is in the imaging volume of the MRI probe, and RF coil 112 is close enough and/or held still enough, to obtain good imaging data from the blood vessel wall.

Alternatively or additionally, the NMR signals are used to obtain non-imaging data, such as NMR spectroscopic data. The magnets and RF coil need not have the configuration shown in FIG. 1A. A number of other configurations for small, self-contained MRI probes have been described, for example in U.S. Pat. Nos. 6,600,319, and 6,704,594, and in patent application Ser. Nos. 10/968,853 and 10/968,828 the disclosures of which are incorporated herein.

Alternatively or additionally, probe 106 has sensing tools for imaging or non-imaging applications other than MRI or NMR, for example for ultrasound imaging, or chemical sensing, or temperature sensing. Alternatively or additionally, probe 106 has tools for therapy, for example RF electrodes for ablating plaque. For all of these applications, balloon 116, when it is expanded, holds the probe in place with one side of the probe (the bottom side in FIG. 1A) close to a wall of the blood vessel, where the sensing or therapeutic tool can be effective. Optionally, for example for chemical sensing, a portion of probe 106 is exposed by an opening in the balloon, without compromising the ability of the balloon to hold pressure. The tool itself need not be pressed against the blood vessel wall. In some applications, for example for focused ultrasound, it may be desirable to use a spacer which holds the tool (in this case an ultrasound transducer) away from the wall.

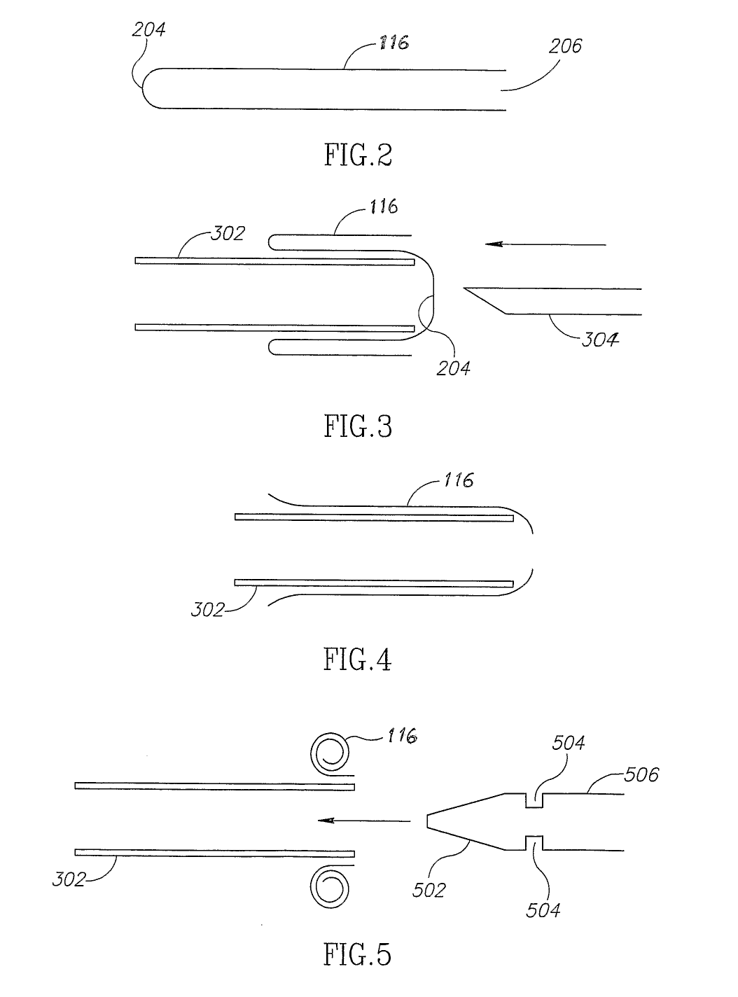

Also shown in FIG. 1A are grooves 118, that go all the way around probe 106 near the proximal and distal ends thereof. Grooves 118 are optional, and may help to seal the balloon against the central portion of the probe. For this end, they may have any combination of wires, epoxy and shrink wrap, which seal the ends of the balloon against the central portion of the probe. Grooves are particularly useful when wire is used to seal the ends of the balloon. When only epoxy and/or shrink wrap is used, grooves may not be so important. The distal groove may be present even if the proximal groove is not, and vice versa. The details of these seals and how they are made will be described below in the description of FIGS. 2-20. Alternatively, other means are used to seal the balloon.

Catheter 108 includes inflation tube 122, the end of which opens up inside balloon 116, as well as one or more cables 124 which carry power and control signals to the RF coil, and convey NMR signals from the RF coil, for example to a controller located outside the body.

Note that, in contrast to most balloons used in angioplasty, which are fairly inelastic and only expand to a more or less fixed size regardless of the pressure used, balloon 116 is highly elastic, and can expand to the diameter of the blood vessel for a range of diameters, depending on the pressure used. For example, the balloon can expand by a factor of 2.7 in diameter, for example from 1.5 mm to 4 mm in diameter, as the internal pressure (above the external pressure) varies from much less than 1 atmosphere to 1 atmosphere, or to 2 atmospheres, or it can expand by a factor of 2, 2.5, 3, or 3.5 in diameter, or by a higher or lower or intermediate factor, with this change in pressure. Optionally, the initial, unexpanded, diameter is 1 mm, or 2 mm, or a higher or lower or intermediate value. Optionally, a higher or lower maximum pressure than 1 or 2 atmospheres is used. However, using a maximum pressure that is no more than 1 or 2 atmospheres has the potential advantage that the balloon is unlikely to rupture the blood vessel. Another advantage of using a very elastic balloon is that it can be much thinner than a typical angioplasty balloon, and hence the magnets and RF coil can be closer to the desired imaging region in the wall of the blood vessel. For example, a typical angioplasty balloon is between 100 and 200 micrometers thick, while balloon 116 is optionally thinner than 30 micrometers. Alternatively, balloon 116 is between 30 and 40 micrometers thick, or between 40 and 60 micrometers thick, or between 60 and 80 micrometers thick, or thicker than 80 micrometers. For an NMR probe which is only 1 mm to 2 mm in diameter, the thickness of the balloon may make a significant difference in the static and RF magnetic field strength that can be obtained in the imaging region, and hence in the signal to noise ratio and resolution that can be obtained in a given acquisition time. Optionally, balloon 116 is designed to function as an angioplasty balloon, as well as functioning to hold probe 106 in place against the blood vessel wall to produce MRI images.

Optionally, when balloon 116 is sufficiently expanded to wedge probe 106 against the walls of the blood vessel, the blood vessel is not completely blocked, and blood can still flow. For example, blood can flow around the sides of the stiff part of the balloon which does not expand. Additionally or alternatively, there is a passageway inside the probe, not shown, which blood can flow through. Additionally or alternatively, the expanded balloon has a cross-section with one or more grooves on the outside that blood can pass through, for example it has a heart-shaped cross-section. Such non-circular cross-sections are produced, for example, by making some azimuthal regions of the balloon relatively stiff, and possibly of fixed curvature azimuthally. In general, blood can flow around the balloon if the probe, with the balloon expanded, has a non-circular cross-section, which does not fill up the blood vessel, and if the balloon does not press hard enough against the blood vessel to significantly distort the shape of the blood vessel.

Alternatively or additionally, instead of coating one side of the balloon with a stiffening material such as parylene, the stiffer side of the balloon is made thicker than the more elastic side.

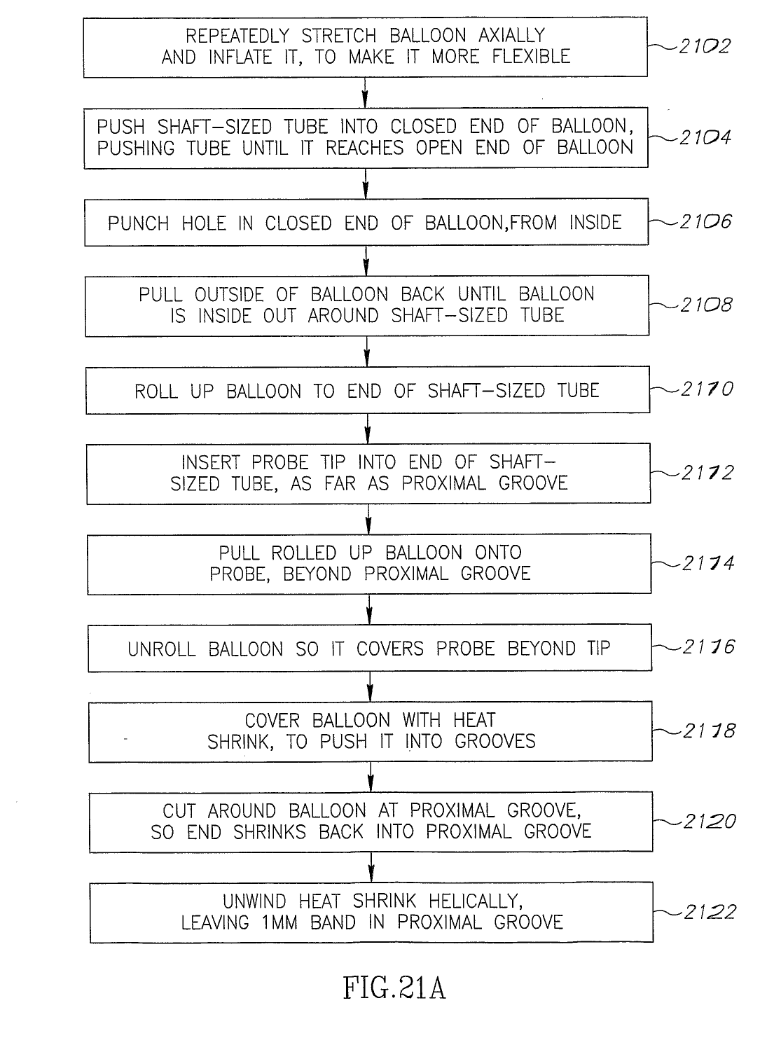

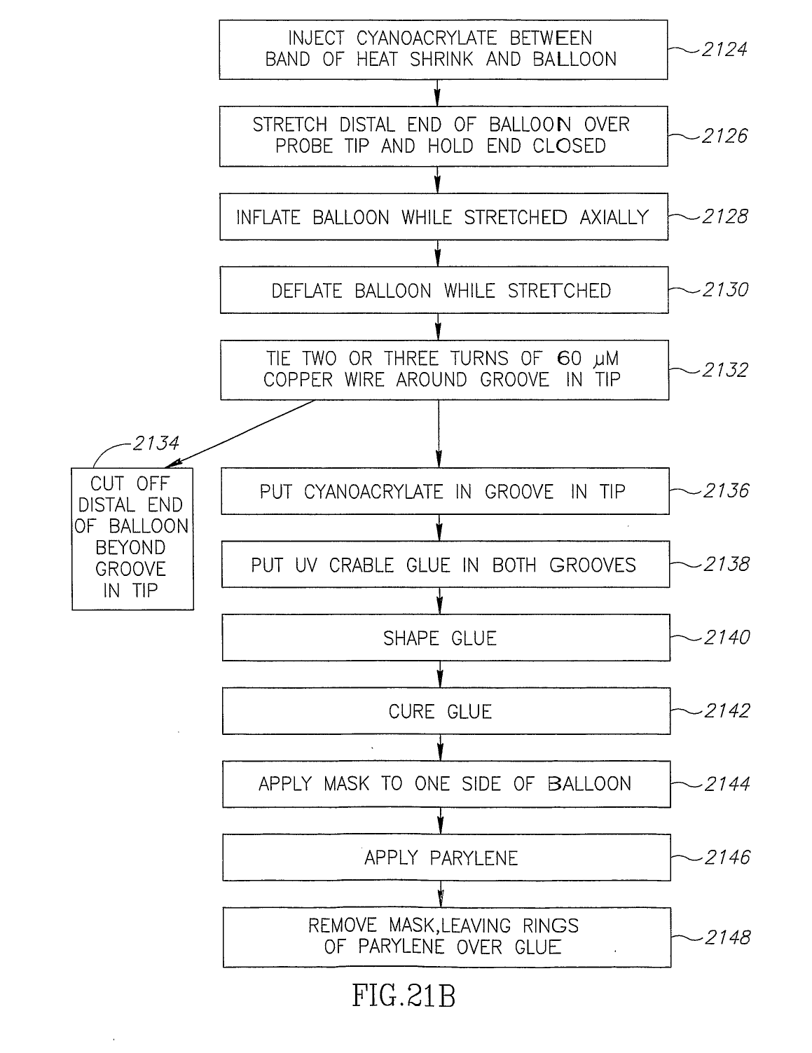

FIGS. 2 through 20 show one possible process by which the balloon is assembled on the MRI probe. The steps illustrated in FIGS. 2 through 20 are listed in a flow chart shown in FIGS. 21A and 21B; the flow chart is divided into two parts because it is too long to fit conveniently on one page. Except as noted, the parts shown in FIGS. 2 through 20 are optionally axisymmetric.

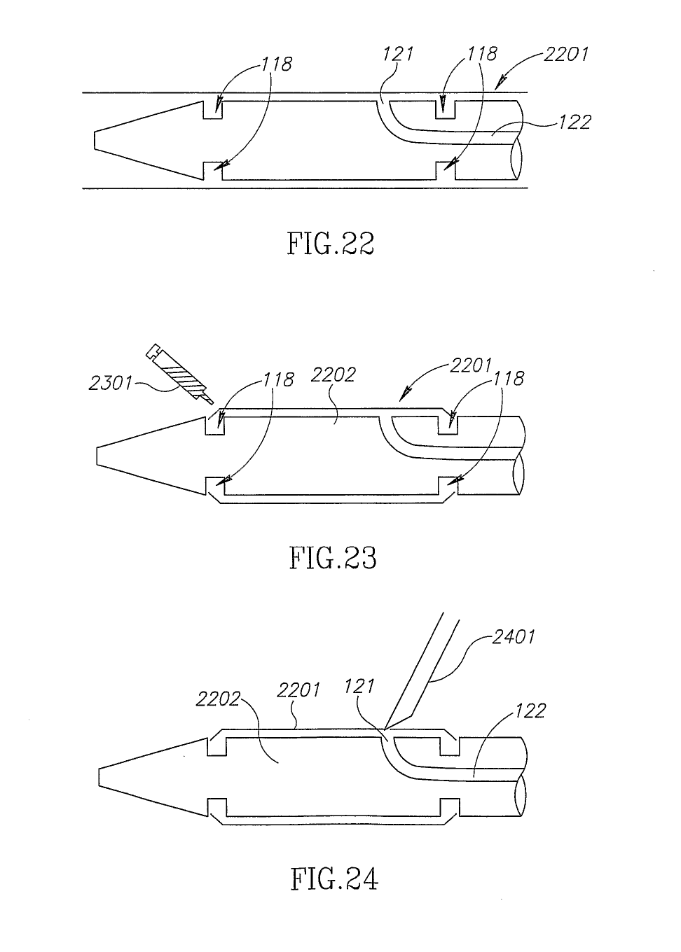

FIG. 2 shows a balloon 116, before assembly. Balloon 116 is made of a biocompatible material if it is intended for use in a biomedical probe, for example silicone, or a blend of silicone and polyurethane, or another biocompatible elastic material. However, as will be explained below, some of the steps in the procedure of assembling the balloon to the probe described in FIGS. 2-19 may not work very well if the balloon is made of a blend of silicone and polyurethane, and may work best with a balloon made of pure silicone. For a balloon made with a blend of silicone and polyurethane, a different assembly procedure, described in FIGS. 22-34, may work better.

The balloon has a diameter such that it will fit snugly around the probe. For example, for a 5.5 French probe, suitable for use in a blood vessel, at least a substantially straight blood vessel, with inner diameter between 2 mm and 4 mm, the balloon optionally has an initial diameter of 1.5 mm. (For a very tortuous blood vessel of this diameter range, a smaller initial probe diameter might be needed.) The balloon is somewhat longer than the probe, since, in some embodiments of the invention, it will be cut during the process of installing it on the probe, and thin enough so that it can expand elastically, i.e. a moderate increase in the pressure, for example from much less than 1 atmosphere up to 1 atmosphere, or 2 atmospheres, will result in a significant increase in expanded diameter, for example from 1.5 mm to 4 mm, without the balloon bursting. A silicone balloon 60 micrometers thick, or a 30 micrometer thick balloon made of a blend of polyurethane and silicone, for example, may be satisfactory for achieving this range of diameters. Different balloon thicknesses may be appropriate for probes with different diameter ranges and lengths, and for different balloon materials. The balloon initially optionally has a closed end 204, and an open end 206. Optionally, before assembling the balloon onto the probe, the balloon is repeatedly stretched axially and inflated, to make it more flexible (2102 in FIG. 21A).

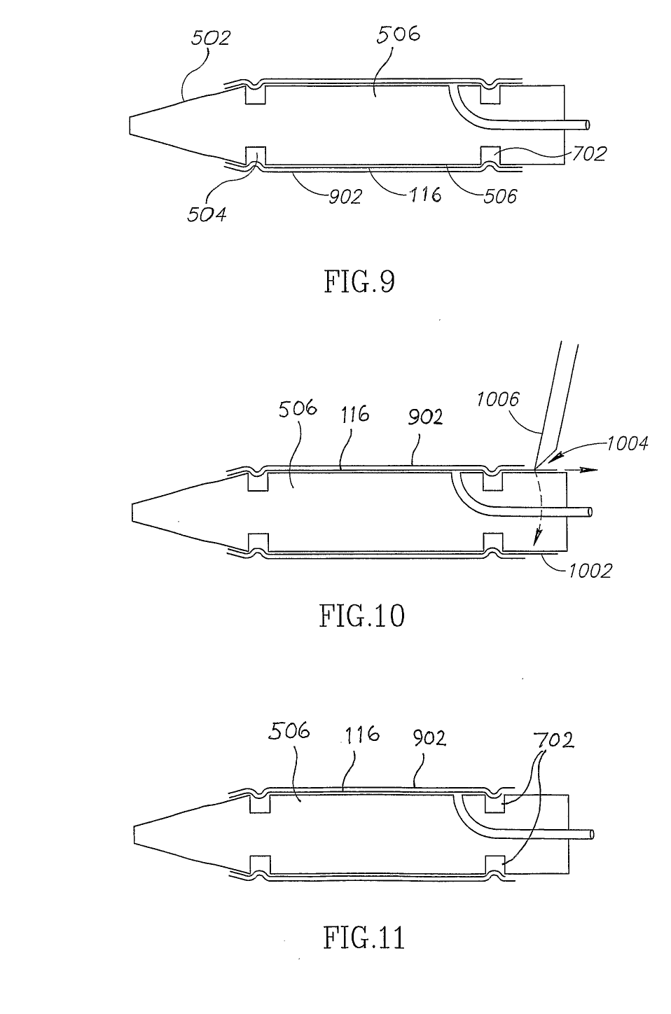

FIGS. 3-8 describe an exemplary method of placing balloon 202 onto the outside of a central portion 106 of the probe. FIGS. 9-12 and 16-19 describe an exemplary method of sealing balloon 202 to the probe, while FIGS. 13 through 15 describe a method of making the balloon more elastic by stretching and inflating it in advance. Optionally, other methods known in the art may be used to accomplish these tasks. For example, FIGS. 26-32 will describe an alternative method of placing the balloon on the central portion of the probe, and stretching and inflating the balloon in advance. FIG. 20 will describe an exemplary method of making the balloon asymmetrically expandable.

As shown in FIG. 3, a tubular tool 302 the same diameter as the central portion of the probe (i.e. the probe before the balloon is installed, optionally including the MRI sensor) and at least as long as the balloon, is pushed into closed end 204 of the balloon until the balloon is half inside-out, with the inside of closed end 204 exposed at open end 206 (2104 in FIG. 21A). A sharp tool 304 is used to punch a hole in closed end 204 (2106 in FIG. 21A), and the rest of the balloon is turned inside out (2108 in FIG. 21A), reaching the state shown in FIG. 4, where balloon 116 is completely inside out on the outside of tool 302. The hole in closed end 204 need not be punched as soon as closed end 204 is accessible from open end 206, but may be punched after the balloon is completely inside out, or any time in between.

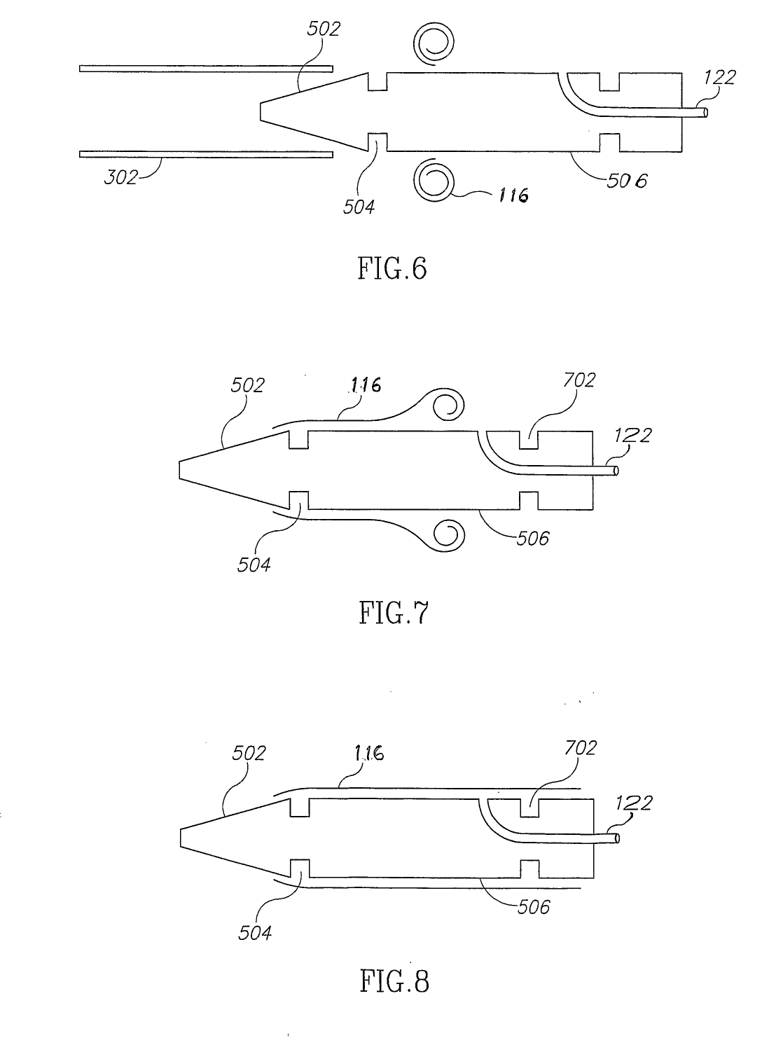

As shown in FIG. 5, balloon 116 is then rolled up to the end of tubular tool 302 (2110 in FIG. 21A). Although a silicone balloon is easily rolled up in this way, it may be difficult to roll up the balloon if it is made of a blend of polyurethane and silicone, and, as noted previously, a different procedure for mounting the balloon to the probe may be advantageous to use in that case. A probe shaft 506 (also referred to previously as the support element, or as the central portion of the probe) is then brought up to the end of tubular tool 302 where rolled up balloon 116 is located. Probe shaft 506 comprises a tapered tip 502, which fits inside tubular tool 302, and a distal groove 504. The dimensions of the groove as shown in FIG. 5 and the other drawings are not necessarily to scale. For a probe that is 1.62 mm in diameter without the balloon expanded, the actual width of the groove is, for example, 0.2 mm or 0.35 mm or 0.5 mm or 0.7 mm or a smaller, larger, or intermediate size, and the actual depth of the groove is 0.2 mm or 0.3 mm or 0.4 mm or a smaller, larger, or intermediate size. The proximal portion of probe shaft 506 is not shown in FIG. 5, but is shown in FIG. 6 and in the following drawings.

As shown in FIG. 6, tip 502 of probe shaft 506 is inserted into the end of tubular tool 302 (2112 in FIG. 21A), adjacent to rolled up balloon 116, and rolled-up balloon 116 is then transferred to probe shaft 506, optionally just past distal groove 504 (2114 in FIG. 21A).

FIG. 7 shows the proximal portion of probe shaft 506, on the right, as well as the distal portion, on the left. The proximal portion comprises a proximal groove 702, and inflation tube 122, also described in FIG. 1A. Inflation tube 122 is not generally axisymmetric, but optionally opens on the upper side of probe shaft 506, as shown in FIG. 7, at a location between the proximal groove and the distal groove. Rolled up balloon 116 is now unrolled over probe shaft 506 (2116 in FIG. 21A), until it extends over both distal groove 504 and proximal groove 702, as shown in FIG. 8. Optionally, instead of unrolling balloon 116 from the distal to the proximate end of the probe, i.e. from left to right as shown in FIGS. 6 and 7, balloon 116 is moved, still rolled up, further toward the proximate end of the probe, i.e. to the right side of the probe as shown in FIGS. 6 and 7, and then unrolled toward the distal end of the probe, i.e. from right to left.

Alternatively, balloon 116 is transferred from tubular tool 302 to probe shaft 506 without rolling the balloon up, or it is placed on probe shaft 506 directly without using tubular tool 302, but the procedure described may make it easier to get balloon 116 onto probe shaft 506.

As shown in FIG. 9, a shrink wrap 902, which may be in the form of a tube or a strip, is optionally placed around probe shaft 506 (2118 in FIG. 21A), covering most of balloon 116, but leaving a small portion of balloon exposed at the proximal end of probe shaft 506. When the shrink wrap is heated and shrunk, balloon 116 is forced into grooves 504 and 702. Alternatively, another means is used to force balloon 116 into the grooves.

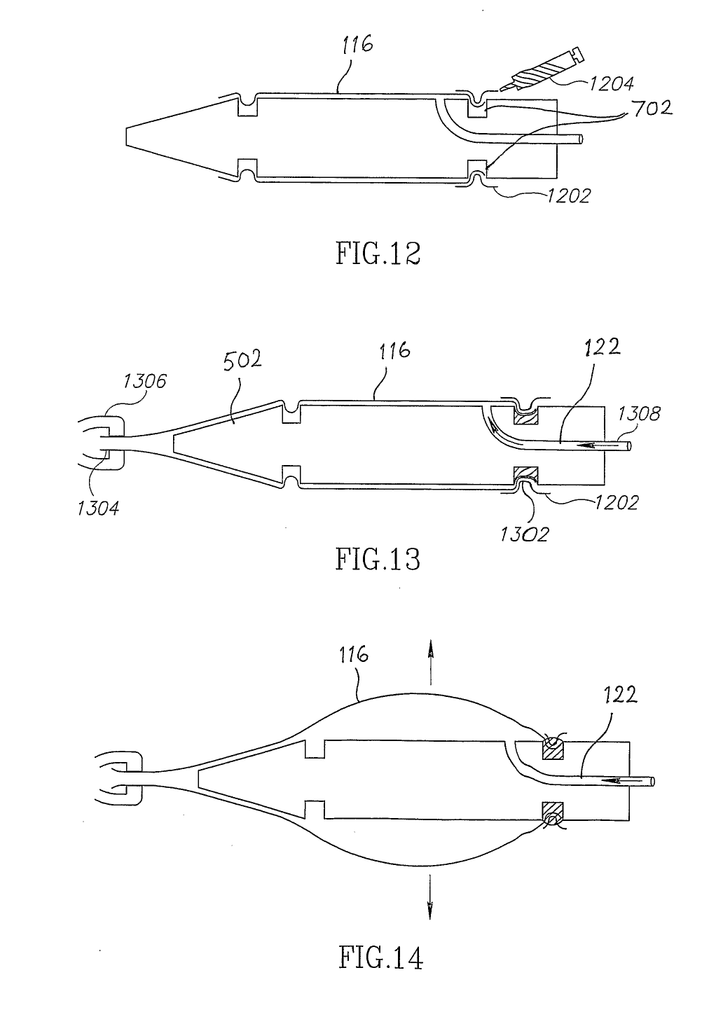

As shown in FIG. 10, the exposed proximal end 1002 of balloon 116 is optionally stretched toward the proximal end of the probe shaft, and, while balloon 116 is stretched, a cut is made all the way around balloon 116, at a location 1004 just beyond the proximal end of the shrink wrap, using a knife 1006 or another cutting tool (2120 in FIG. 21A). Balloon 116 then shrinks back under shrink wrap 902, with its proximal end inside proximal groove 702, as shown in FIG. 11. Shrink wrap 902 is then optionally unwound helically from probe shaft 506 (2122 in FIG. 21A), starting from the distal end, leaving a band 1202 of shrink wrap about 1 mm wide around proximal groove 702, as shown in FIG. 12.

As shown in FIG. 12, a cyanoacrylate 1204, or a glue with similar properties, is optionally injected under the band of shrink wrap (2124 in FIG. 21B). Surface tension draws the cyanoacrylate into groove 702, including under balloon 116. Although hardened cyanoacrylate 1302 is shown in FIG. 13 as having a finite volume, for clarity, in fact the cyanoacrylate is applied and dries in a very thin layer, drawing shrink wrap band 1202 into close contact with balloon 116, and drawing balloon 116 into close contact with groove 702, providing a pressure-tight seal.

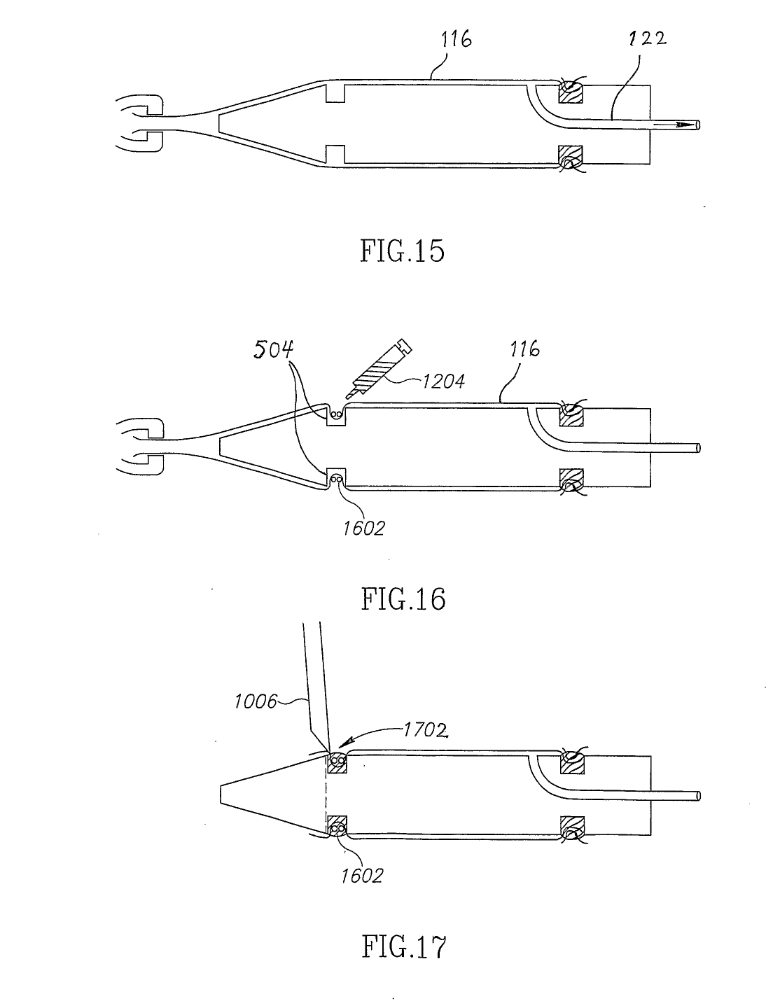

The distal end 1304 of balloon 116 is then optionally stretched axially over probe tip 502, as shown in FIG. 13, and the distal end is held closed while it is stretched, for example using pliers 1306, or a similar tool (2126 in FIG. 21B). Pressurized air or another pressurized fluid is then optionally injected through inflation tube 122, to inflate the balloon while it is stretched axially (2128 in FIG. 21B), as shown in FIG. 14, and the balloon is optionally stretched further. The pressure is then released to deflate the balloon, optionally while it is still stretched (2130 in FIG. 21B), as shown in FIG. 15. This inflation and stretching of the balloon optionally serves to make it thinner, so that there is less distance between the MRI probe and the wall which may make the elasticity of the balloon more predictable, and may increase the diameter to which the balloon can expand without breaking. Optionally, a different procedure for inflating and stretching the balloon is used instead of, or in addition to, the procedure described here. For example, the procedure described below, in the description of FIGS. 26-32, is used.

Without releasing the balloon from its axial tension, as shown in FIG. 16, two or three turns of wire 1602, for example copper wire of 60 micrometer diameter, are optionally wrapped tightly around the outside of balloon 116 over distal groove 504 (2132 in FIG. 21B), forcing the balloon further into the distal groove. Cyanoacrylate 1204, or a similar glue, is then optionally injected into groove 504 (2134 in FIG. 21B), where it is drawn between wire 1602 and balloon 116. As in the case of the cyanoacrylate in proximal groove 702, hardened cyanoacrylate 1702 in distal groove 504 is shown in FIG. 17 as having finite thickness, but in fact it forms a very thin layer, holding wire 1602 firmly against balloon 116, and holding the turns of wire 1602 against each other, in distal groove 504, thereby providing a pressure-tight seal between balloon 116 and distal groove 504. The distal end of balloon 116 is then released from tension, and knife 1006 or a similar cutting tool is optionally used to make a cut around the balloon at location 1704, just beyond the distal groove (2136 in FIG. 21B). Optionally, the cut is made before injecting the cyanoacrylate. Alternatively, another means is used to seal the balloon against groove 702.

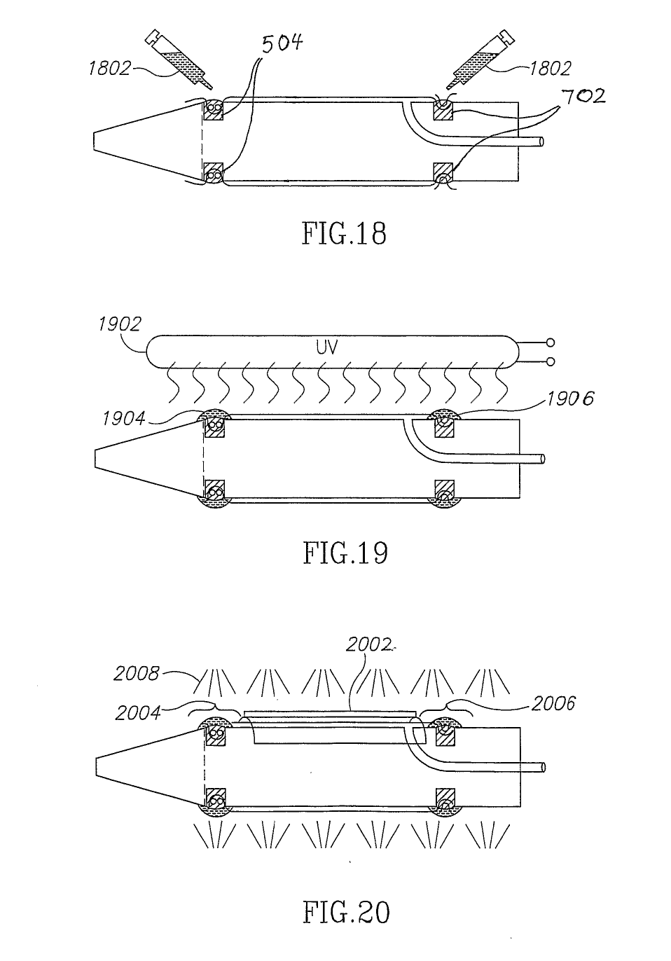

As shown in FIG. 18, a UV curable glue 1802 is then optionally injected into both the distal and proximal grooves (2138 in FIG. 21B), and shaped (2140 in FIG. 21B). For example, the glue is optionally shaped so that, when it hardens, the surface of the glue forms a continuous smooth surface connecting the balloon with the probe shaft surface beyond the grooves. The round shapes shown in FIG. 19 for the shaped glue masses 1904 in the distal groove, and 1906 in the proximal groove, are merely illustrative. As shown in FIG. 19, a UV lamp 1902 is used to harden the UV glue, once it has been shaped (2142 in FIG. 21B).

As shown in FIG. 20, a mask 2002 is optionally placed around the upper part of the probe (2144 in FIG. 21B). Mask 2002 is not axisymmetric, but covers only a part of the balloon azimuthally (a part that is on top in FIG. 20). The bottom of the balloon is not covered, and a portion 2004 of the probe including the distal groove, and a portion 2006 of the probe including the proximal groove, are optionally kept uncovered all the way around the probe.

A stiffening agent 2008, for example parylene, is then deposited on the probe (2146 in FIG. 21B) using vapor deposition, or any other technique known in the art of depositing thin layers of material. The mask is then removed (2148 in FIG. 21B), and the part of the balloon under the mask is free of parylene, and remains flexible, while the unmasked part of the balloon becomes hardened and much less flexible. In addition, bands of parylene cover the distal and proximal grooves going all the way around the probe, which prevents the UV glue from cracking. Although the UV glue could be omitted, it is potentially stronger than parylene, and it may be easier to form into a built-up shape than parylene, so the combination of UV glue and parylene is potentially better than either one by itself. Optionally, parylene is also deposited on some locations of the expanding parts of the balloon, for example to shape the cross-sectional shape of the balloon.

Alternatively, instead of or in addition to using a mask, another means is used to confine the parylene coating (or whatever hardening agent is used) to only part of the balloon surface. For example, the stiffening agent is sprayed on from one direction, or painted on, or otherwise applied from one direction. If the probe is rotated while the stiffening agent is sprayed on, and it is rotated faster when it is in some orientations than in other orientations, and/or if it moves closer to and further away from the source of the spray depending on its orientation, then the stiffening agent will be thicker in some places than in other places.

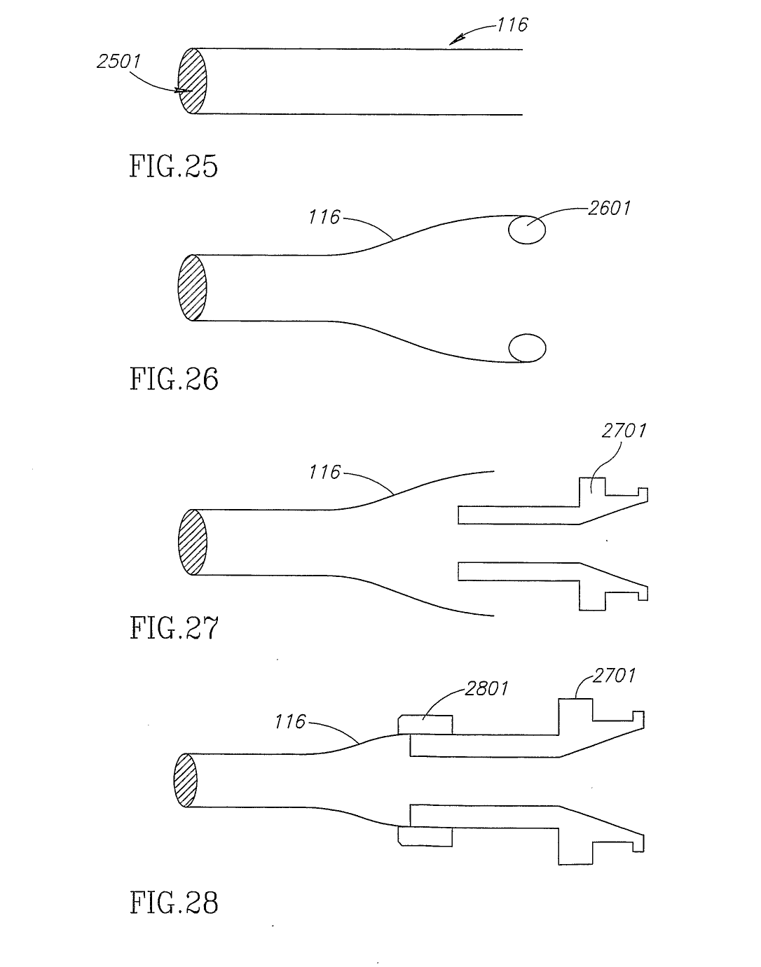

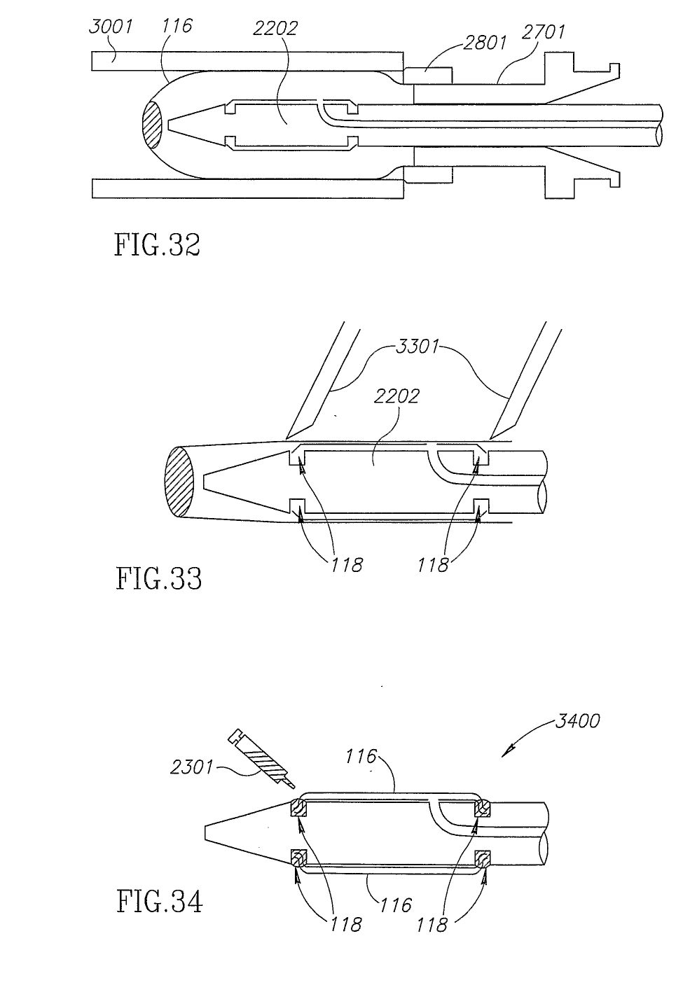

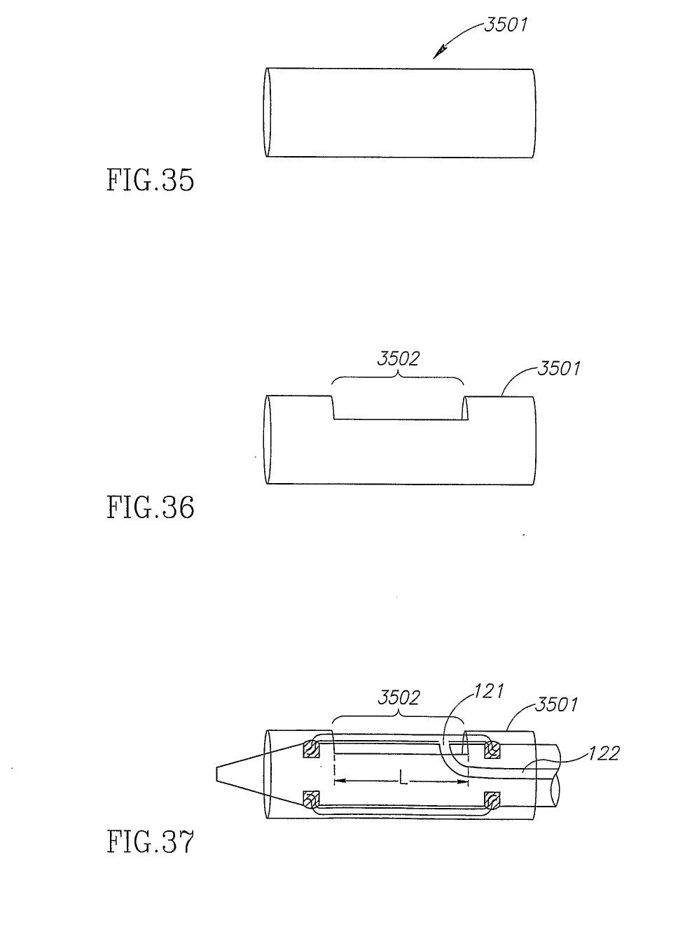

FIGS. 22-39 show procedures for assembling a probe with alternative designs for an asymmetrically expanding balloon, similar to probe 106 and balloon 116 in FIGS. 1A and 1B, according to an exemplary embodiment of the invention. FIGS. 22-24 show an initial preparation of the probe, before assembling the balloon to the probe. FIGS. 25-31 show an initial preparation of the balloon, and FIGS. 32-34 show the assembly of the balloon to the probe. FIGS. 35-39 show procedures for making the balloon asymmetrically expandable, according to some exemplary embodiments of the invention. Other methods of making the balloon asymmetrically expandable, after it has been assembled to the probe, will also be described.

As shown in FIG. 22 a layer 2201 of shrink wrap (for example PET shrink 850025, sold by Advanced Polymers) is placed around a probe shaft 2202, similar to probe shaft 506 in FIG. 6. A strip of shrink wrap is wound around the probe shaft helically, for example, or the probe shaft is placed inside a closely fitting tube of the shrink wrap. Shrink wrap 2201 is optionally somewhat longer than probe shaft 2202. Probe shaft 2202 optionally has distal and/or proximal grooves 118, and has an inflating tube 122 with an outlet 121.

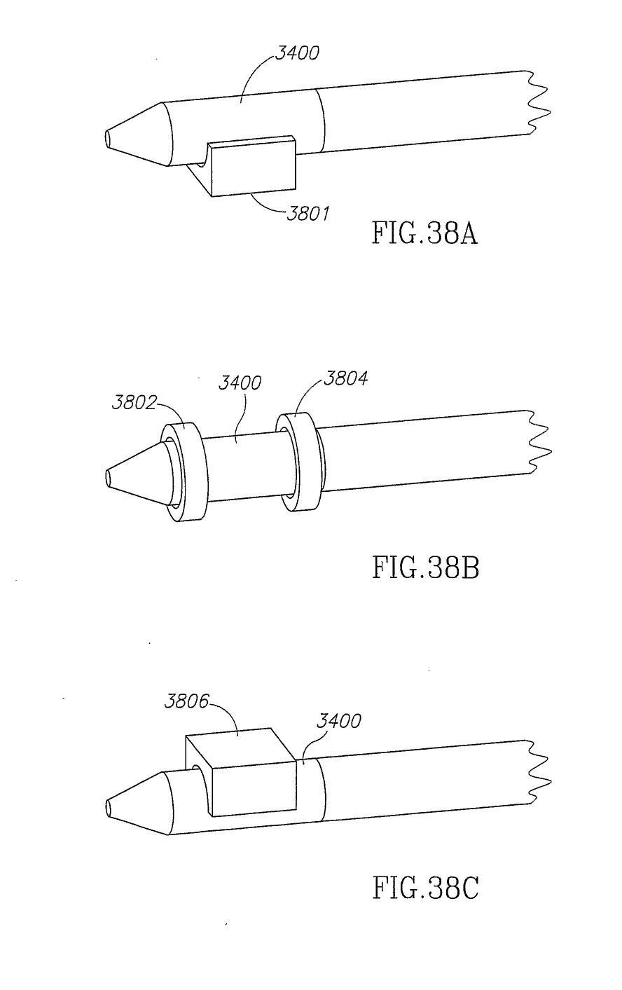

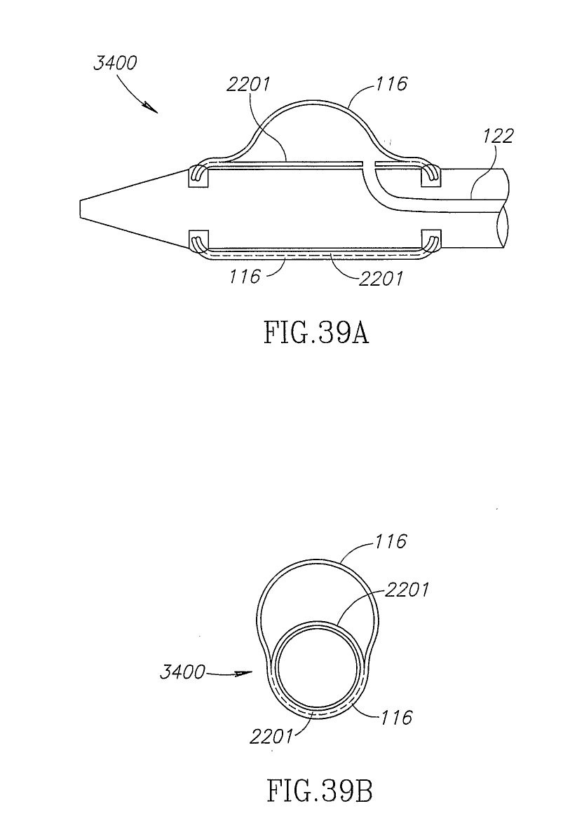

In an embodiment of the invention described below in FIGS. 38 and 39, layer 2201 optionally comprises an elastomeric material, instead of shrink wrap. In this case, optionally, a ring of shrink wrap is placed around layer 2201 at each of two axial locations, for example at the locations of grooves 118, and then shrunk, in order to hold layer 2201 to probe shaft 2202. Glue, for example cyanoacrylate, is then optionally applied between the rings of shrink wrap and layer 2201, and/or between layer 2201 and probe shaft 2202. However, this procedure works best when the elastomeric material is a material, such as a blend of polyurethane and silicone, that sticks well to glue, and may not work at all if the elastomeric material is pure silicone, which tends not to stick very well to glue.

As shown in FIG. 23, after shrink wrap 2201 is shrunk around probe shaft 2202 it is optionally shortened to extend only between distal and proximal grooves 118. This may be done, for instance, by cutting off portions of the shrink wrap that extend beyond the grooves. Glue, for instance cyanoacrylate, is optionally applied under the shrink wrap on both distal and proximal grooves 118 by an applicator 2301, for example in order to prevent unintentional unwinding and/or help in sealing the wrap to the probe.

It should be noted that, although probe shaft 2202 is shown in FIGS. 22-34 and FIGS. 36-37 as having grooves 118, it may not be necessary or even useful to have grooves in this embodiment of the invention, since the balloon is not sealed to the probe using wires. If no grooves are present, then various procedures described as taking place at the grooves instead take place at the surface of the probe shaft, at the same axial locations where the grooves are shown in the drawings.

As shown in FIG. 24 the shrink wrap is then pierced with a needle 2401 (for example a 30 G needle) above outlet 121 of inflation tube 122, so that later, after the balloon is assembled to the probe, it will be possible to expand the balloon using inflation tube 122.

As shown in FIG. 25 a tube 116 made of an elastomeric material, is sealed at one of its ends with a sealing 2501. This may be achieved, for instance, using UV-curing glue (for example 8106-1 glue sold by Panacol-Elosol). Tweezers can be used to squeeze tube 116 shut, to prevent glue capillarity into the tube. In an embodiment of the invention described below in FIGS. 38 and 39, layer 2201 is optionally made of the same elastomeric material as tube 116.

Suitable materials for tube 116 include the blends of polyurethane and silicone sold by Cardiotech International under the registered trademark Polyblend 1100, in particular type 60A, with hardness of Shore 60, which the inventors have tested. Type 45, with a hardness of Shore 45, as well as other types, might also be satisfactory. Although tube 116 is also optionally made of silicone, using a blend of polyurethane and silicone has the potential advantage that it can be made thinner while still expanding safely to a same diameter, thereby allowing probe shaft 2202 to get closer to a blood vessel wall for example, which can improve the signal to noise ratio if, for example, probe shaft 2202 is an MRI probe looking at plaque in the blood vessel wall. Another potential advantage of using a blend of polyurethane and silicone is that the balloon may be sealed by gluing it to an underlying layer of shrink wrap, while a pure silicone balloon, which is not glued so easily, may have to be sealed using wires and/or shrink wrap, as described in FIGS. 9-19.

Using reverse action tweezers 2601 as shown in FIG. 26 the tube 116 is optionally expanded at its open end, and a pump nozzle 2701 is optionally inserted to the open end of the tube as shown in FIG. 27, in order to expand and stretch the balloon, as will be explained below.

As shown in FIG. 28 a gasket 2801 is optionally placed around the tip of nozzle 2701, holding the open end of the tube expanded. Gasket 2801 is of dimensions and made of a material, for example, such that it holds tube 116 tightly to nozzle 2801, making a reasonably good vacuum seal. A rubber gasket of a type used in a homeostatic valve may be satisfactory, for example.

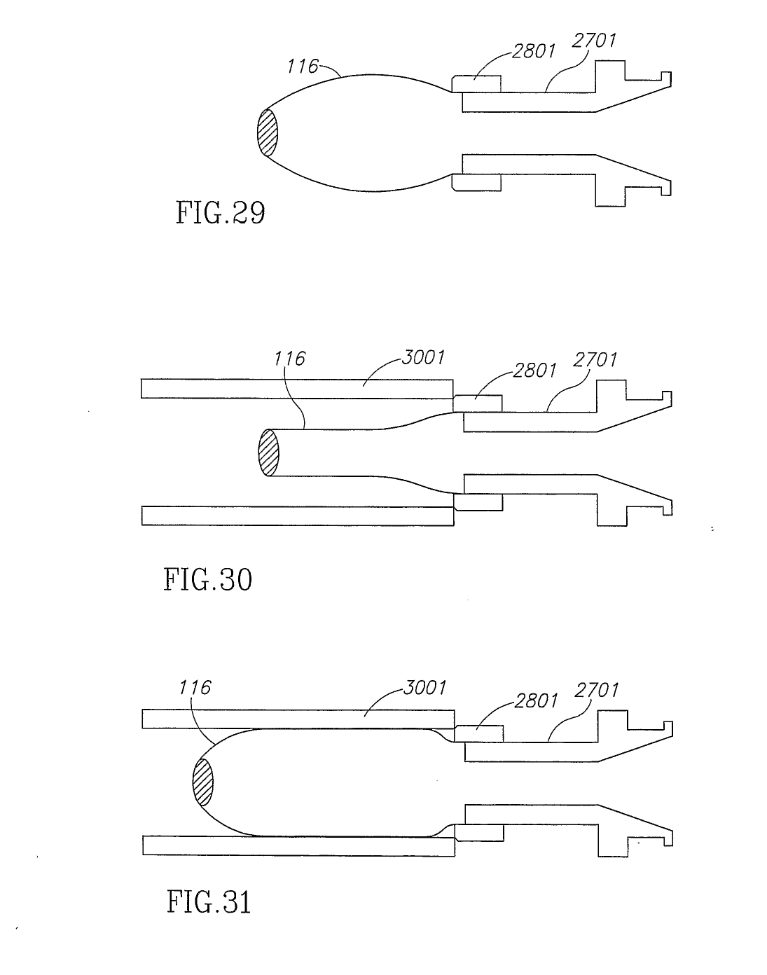

As shown in FIG. 29, tube 116 is optionally inflated until its whole length expands, by pumping air or another fluid into tube 116 through nozzle 2701. Expanding tube 116 in this way has the potential advantage of increasing the limit of the tube's elasticity. In the case where the tube is made of Polyblend 1100, type 60A, it is advantageous to apply a pressure of up to 0.8 Bar, for example, and then keep the tube with a pressure of 0.5 Bar for 30 seconds.

As shown in FIG. 30 a rigid vacuum hose 3001, optionally transparent, is placed against gasket 2801.

As shown in FIG. 31, when vacuum hose 3001 is activated, tube 116 expands inside the vacuum hose.

As shown in FIG. 32, probe shaft 2202, prepared as described in FIGS. 22-24, is inserted into tube 116. Vacuum hose 3001 is then brought back to ambient pressure. Gasket 2801 and the nozzle of vacuum hose 3001 may then be removed.

As shown in FIG. 33, while using a knife 3301 or another sharp tool both sides of tube 116 are cut (using the same procedure described in the description of FIG. 23, for example) near grooves 118.

As shown in FIG. 34, tube 116 is sealed to the probe, in a pressure-tight seal, at grooves 118, using cyanoacrylate or other suitable glue, as described above in the description of FIG. 23. The resulting probe, labeled 3400 in FIG. 34, is used in several different embodiments which will be described now.

According to one such embodiment, the balloon is now given asymmetric expandability by selectively applying to it a stiffening material, for instance, as described in relation to FIG. 20 above. It should be noted that applying a parylene coating, as described in FIG. 20, is typically done as a batch process, in which a large number of probes, for example 20 or 30 probes, are put in a chamber and the coating is applied to all of them simultaneously. Using a batch process has the potential advantage that it may be less labor intensive than if each probe is processed individually. On the other hand, processing each probe individually has the potential advantage that, if something goes wrong with the process, no more than one probe is ruined, and it may be possible to correct problems with the process before any additional probes are ruined. FIGS. 35-39 describe embodiments of the invention in which each probe is processed individually.

According to one embodiment of the invention, as shown in FIG. 35, a sheet of shrink wrap 3501, for example PET shrink wrap, is flattened and cut somewhat longer then the probe.

As shown in FIG. 36, shrink wrap 3501 is cut in a rectangular shape with an opening 3502. As shown in FIG. 37, the length L of the opening is somewhat shorter than the distance between distal and proximal grooves 118. Cut shrink wrap 3501 is placed on the probe such that outlet 121 of inflating tube 122 is not covered by shrink wrap 3501, but is within opening 3502.

Shrink wrap 3501 is then shrunk and its edges are cut and sealed, for example in the same way as shrink wrap 2201 as described in FIG. 23 or tube 116, as described in FIGS. 33-34.

According to another embodiment of the invention, probe 3400, shown in FIG. 34, is optionally made using an elastomeric material for inner layer 2201, for example the same material used for tube 116, rather than using shrink wrap for inner layer 2201. After probe 3400 is assembled to the state shown in FIG. 34, inner layer 2201 is selectively bonded to tube 116 at some locations, for example on the side of probe 3400 that is on the bottom in FIG. 34, and around both grooves 118. But layer 2201 is not bonded to tube 116 at other locations, for example on the side of probe 3400 that is on the top in FIG. 34, and in particular not at the opening to inflation tube 122. Optionally, layer 2201 is bonded to tube 116 by heat fusing. Making layer 2201 out of the same elastomeric material as tube 116 has the potential advantage that it may be relatively easy to create a strong bond between tube 116 and layer 2201 by heat fusing.

FIGS. 38A-38C show methods of heat fusing layer 2201 to tube 116. In FIG. 38A, probe 3400 is placed on a heater 3801, which is shaped to heat one side of probe 3400. In FIG. 38B, two ring-shaped heaters 3802 and 3804 are placed around probe 3400, adjacent to grooves 118. Alternatively, only one ring-shaped heater 3802 is used, and is first placed around 3400 adjacent to one of grooves 118, to heat fuse layer 2201 to tube 116 at that end of probe 3400, and then moved to be adjacent to the other groove 118, to heat fuse layer 2201 to tube 116 at that end of probe 3400.

FIG. 38C shows another method of heat fusing layer 2201 to tube 116. A heat shield 3806, for example a ceramic heat shield, is shaped to cover one side of probe 3400. Heat shield 3806 is placed against the side of probe 3400 that includes the opening of inflation tube 122, i.e. the side of probe 3400 that is on the top in FIG. 34. Heat shield 3806 does not cover regions of probe 3400 adjacent to grooves 118. Probe 3400 is then heated from all sides, for example in an oven, and layer 2201 fuses to tube 116, except for the part of layer 2201 and tube 116 which are under heat shield 3806.

FIGS. 39A and 39B schematically show a side cross-sectional view (FIG. 39A) and an axial cross-sectional view (FIG. 39B) of probe 3400, after layer 2201 has been heat fused in some places to tube 116. Tube 116 is shown inflated, where it is not fused to layer 2201.

It should be understood that not all features shown in the drawing or described in the associated text may be present in an actual device, in accordance with some embodiments of the invention. Furthermore, variations on the method and apparatus shown are included within the scope of the invention, which is limited only by the claims. Also, features of one embodiment may be provided in conjunction with features of a different embodiment of the invention. As used herein, the terms “have”, “include” and “comprise” or their conjugates mean “including but not limited to.”

1. A device adapted to be inserted into a lumen, the device having a longitudinal axis and comprising:
a) a support element extending along the longitudinal axis;
b) a tool mechanically mounted on the support element and being adapted to be used near a wall of the lumen on at least a first side of the longitudinal axis; and
c) element, the balloon having at least one portion that is less radially expandable than at least one other portion of the balloon, at a same axial position along said support element.

2. A device according to claim 1, wherein the tool comprises an MRI coil.

3. A device according to claim 1, wherein the tool comprises MRI coil and magnet.

4. A device according to claim 1, wherein the support element is rigid.

5. A device according to claim 1, wherein the balloon surrounds the tool and the support element.

6. A device according to claim 1 wherein the lumen is in the body.

7. A device according to claim 6, wherein the lumen is a portion of the digestive track.

8. A device according to claim 7, wherein the lumen is the rectum.

9. A device according to claim 6, wherein the lumen is a blood vessel.

10. A device according to claim 9, wherein the device with a non-expanded balloon has an outer diameter of less than 1 mm

11. A device according to claim 9, wherein the device with a non-expanded balloon has an outer diameter of less than 2 mm.

12. A device according to claim 9, wherein the device with a non-expanded balloon has an outer diameter of between 2 mm and 4 mm.

13. A device according to claim 1, wherein the tool is configured to perform a diagnostic task.

14. A device according to claim 13, wherein diagnostic task comprises information gathering by NMR.

15. A device according to claim 13, wherein the diagnostic task comprises imaging.

16. A device according to claim 13, wherein the diagnostic task comprises detecting plaque in a blood vessel.

17. A device according to claim 13, wherein the diagnostic task comprises diagnosing one or more characteristics of plaque in a blood vessel.

18. A device according to claim 1, wherein the balloon safely inflates to a range of sizes varying by at least a factor of 2.

19. A device according to claim 18, wherein the range includes 3 mm.

20. A device according to claim 1, wherein the balloon comprises silicone.

21. A device according to claim 20, wherein the balloon comprises a blend of silicone and polyurethane.

22. A device according to claim 1, wherein the balloon is thicker on the first side of the axis.

23. A device according to claim 1, wherein the balloon is coated with a stiffening material on at least the first side of the axis.

24. A device according to claim 23, wherein at least a portion of the balloon on the side of the axis opposite the first side is not coated with the stiffening material.

25. A device according to claim 23, wherein the stiffening material comprises parylene.

26. A device according to claim 1, wherein a heat shrink material is applied to a balloon's portion that is less expandable than other portions of the balloon.

27. A device according to claim 26, wherein the heat shrink material comprises PET.

28. A device according to claim 1, also including a sealing element which holds the balloon against the support element in a pressure-tight manner.

29. A method of manufacturing a device adapted to be inserted into a lumen, the method comprising:
a) placing an elastically expandable balloon around a support element having an axis;
b) masking the balloon asymmetrically, such that a first portion of the balloon is masked more than a second portion of the balloon, said first and second portions extending along the axis.
c) applying a stiffening material to the masked balloon; and
d) unmasking the balloon.

30. A method according to claim 29, wherein the differences in amount of stiffening material applied to different portions of the balloon allow differences in expandability of such different portions, such that an inflated balloon has one portion that is expanded at least 35% more than another.

31. A method according to claim 29, wherein applying a stiffening material comprises vapor depositing the stiffening material onto the masked balloon.

32. A method according to claim 29, wherein the stiffening material is parylene.

33. A method of manufacturing a device adapted to be inserted into a lumen, the method comprising:
a) placing an elastically expandable balloon around a support element having an axis;
b) applying a heat-shrink material to the first portion of the balloon; and
c) heating the heat-shrink material as to let it shrink around the first portion of the balloon.

34. A method according to claim 29, also including sealing the balloon against the support element.

35. A method according to claim 34, wherein sealing comprises applying glue.

36. A method according to claim 35, wherein the glue comprises a cyanoacrylate.

37. A method according to claim 35, wherein the glue comprises a UV-curable glue.

38. A method of manufacturing a device adapted to be inserted into a lumen, the method comprising:
a) fixing a sheet of polymeric material around a support element having an axis;
b) piercing the sheet of polymeric material as to allow an inflation tube extending along the support element to supply an inflating fluid through the pierce,
c) placing an elastic balloon around the pierced sheet of polymeric material; and
d) heat-fusing a first portion of the elastic balloon to an adjacent portion of the pierced sheet of polymeric material, such that upon supplying inflating material through the inflation tube, the first portion of the balloon will not inflate.

39. A method according to claim 38, wherein the sheet of polymeric material comprises a blend of silicone and polyurethane.

40. A method according to claim 38, wherein the sheet of polymeric material and the elastic balloon are made of the same material.

41. A method of performing a diagnostic task on the wall of a lumen in the body, comprising:
a) inserting a device according to claim 1 into the lumen;
b) expanding the balloon of the device;
c) performing one or both of a diagnostic and therapeutic task on the wall of the lumen on the first side of the axis, using the device.

42. A method according to claim 41, wherein the diagnostic task comprises diagnostic imaging.

43. A method according to claim 41, the diagnostic task comprises diagnostic NMR.

44. A method according to claim 41, wherein the lumen is a blood vessel.

45. A method according to claim 44, wherein the diagnostic task comprises detecting plaque.

46. A method according to claim 44, wherein the diagnostic task comprises diagnosing one or more characteristics of plaque.

47. A device comprising an asymmetric balloon, MRI coil and magnet.
